(19) United States
(12) Patent Application Publication
(10) Pub. No.: US 2025/0009978 A1
(43) Pub. Date: Jan. 9, 2025

(54) METHOD PERFORMABLE WITH AN ELECTRONIC DEVICE IN ORDER TO COMMUNICATE WITH A DRUG DELIVERY DEVICE, ELECTRONIC DEVICE AND DRUG DELIVERY DEVICE

(71) Applicant: Sanofi, Paris (FR)

(72) Inventors: Stefan Alt, Frankfurt am Main (DE); Michael Helmer, Frankfurt am Main (DE)

(21) Appl. No.: 18/705,403

(22) PCT Filed: Nov. 1, 2022
(86) PCT No.: PCT/EP2022/080415

§ 371 Date: Apr. 26, 2024

(30) Foreign Application Priority Data
Nov. 3, 2021 (EP) 21315212.7

(51) Int. Cl. A61M 5/315; A61M 5/20

(52) U.S. Cl. CPC A61M 5/31566; A61M 5/20; A61M 2205/276; A61M 2205/3584; A61M 2205/3592; A61M 2205/6009

(57) ABSTRACT

A method performable with an electronic device in order to communicate with a drug delivery device is described. The method includes determining whether a user of the electronic device is authorized to operate the drug delivery device based on first information and second information. The first information is indicative for a prescription of the drug to a patient and the second information is indicative for the drug the drug delivery device is foreseen to dispense. The method further includes a step of generating an output signal if the user is authorized to use the drug delivery device, wherein the output signal is foreseen to be communicated to the drug delivery device in order to enable changing an operational state of the drug delivery device.

### CROSS-REFERENCE TO RELATED APPLICATIONS

The present application is the national stage entry of International Patent Application No. PCT/EP2022/080415, filed on Nov. 1, 2022, and claims priority to Application No. EP 21315212.7, filed on Nov. 3, 2021, the disclosures of which are incorporated herein by reference.

### TECHNICAL FIELD

A method performable with an electronic device in order to communicate with a drug delivery device is provided. Furthermore, an electronic device, a computer program, a computer readable data carrier and a drug delivery device are provided.

### BACKGROUND

Administering an injection is a process which presents a number of risks and challenges for users and healthcare professionals, both mental and physical. A drug delivery device may aim to make self-injection easier for patients. A safe operation of the drug delivery device is desirable.

### SUMMARY

The disclosure describes an improved method performable with an electronic device in order to communicate with a drug delivery device, particularly a method which increases the safety for the user trying to operate the drug delivery device. Further, the disclosure describes a drug delivery device with which the method can be performed, a computer program and a computer-readable data carrier for performing the method and an improved drug delivery device, particularly a drug delivery device configured to communicate with the electronic device. First, the method is specified.

According to at least one embodiment, the method is performable or is performed with an electronic device. The method is, e.g. performed in order to communicate with a drug delivery device, particularly in order to enable an operation of the drug delivery device. The electronic device may comprise at least one processor. The electronic device may be a computer or a tablet-PC or a smartphone or a smartwatch. Particularly, the method is a computer implemented method.

The drug delivery device and the electronic device are separate devices. For example, the drug delivery device and the electronic device are configured to communicate wirelessly with each other.

According to at least one embodiment, the method comprises a step in which it is determined whether the user of the electronic device is authorized to operate the drug delivery device.

According to at least one embodiment, determining whether the user of the electronic device is authorized to operate the drug delivery device is done based on first information and second information. The first information and the second information are, in particular, digitalized information processable with the electronic device.

According to at least one embodiment, the first information is indicative for a prescription of a drug to a patient. The prescription may be issued by a doctor and the first information about this prescription may have been transmitted to the electronic device.

According to at least one embodiment, the second information is indicative for the drug the drug delivery device is foreseen to dispense. Particularly, the drug delivery device is configured to perform a dispense process in order to dispense a drug dose. The drug delivery device may comprise or may be configured to receive a medicament container containing the drug.

In order to determine whether the user of the electronic device is authorized to operate the drug delivery device, the first information and the second information may be compared to each other and/or to the identity of the user. For example, it may be determined whether the drug prescribed to the patient is the same as the drug which the drug delivery device is foreseen to dispense. Furthermore, it may be determined whether the user of the electronic device is the patient, the drug has been prescribed to. If both conditions (prescribed drug is the drug of the drug delivery device and user of the electronic device is the patient) are fulfilled, it may be determined that the user of the electronic device is authorized to operate the drug delivery device. If at least one of the two conditions is not fulfilled, it may be determined that the user is not authorized to operate the drug delivery device.

For determining whether the user is indeed the patient, the identity of the user may be used and compared to the information about the patient stored in the first information. For this purpose, the user may be assumed to be the owner of the electronic device. For example, the identity of the owner of the electronic device may be electronically stored on the electronic device and this identity may be compared to the identity of the patient. Alternatively, it may be determined whether the prescription was indeed foreseen for this specific electronic device and if this is the case, it may be determined that the user/owner is the patient.

As an optional step, the identity of the user of the electronic device is determined before determining whether the user is authorized. This may be done, e.g., based on a fingerprint, face scan or password insertion. Thus, the electronic device may be configured to determine the identity of the user using face scan, fingerprint or password identification.

Determining whether the user of the electronic device is authorized to operate the drug delivery device may additionally be based on a dosage schedule in order to avoid that the user administers himself an overdosage or a wrong dosage. For example, the first information is indicative for the dosage schedule or third information stored on the electronic device may be indicative for the dosage schedule.

By way of example, the prescription is only valid for a certain time window and only in this time window an output signal for a certain drug delivery device can be generated. Additionally or alternatively, the generation of output signals for different drug delivery devices is constrained by the dosage schedule. For example, two subsequent output signals can only be generated with a predetermined or prescribed time gap between the two signals. The time gap may be several hours.

According to at least one embodiment, the method comprises a step in which an output signal is generated if the user is authorized to use the drug delivery device, i.e. if it has been determined that the user is authorized to use the drug delivery device. If it has not been determined that the user is authorized to operate the drug delivery device, the output signal is, e.g., not generated.

According to at least one embodiment, the output signal is foreseen to be communicated or transferred to the drug delivery device in order to enable changing an operational state of the drug delivery device. The output signal is, e.g., sent from the electronic device with help of a communication component of the electronic device. Particularly, the output signal comprises information which are indicative for the user being authorized to use the drug delivery device. The drug delivery device may be configured to receive the output signal, to extract and/or process and/or understand this information, and then accordingly enable changing the operational state.

The change of the operational state may, in particular, be a change from a state in which a dispense process for dispensing a drug dose and/or a setting process for setting a drug dose is prevented to a state in which the dispense process and/or the setting process is enabled. Accordingly, the user may only perform the desired dose setting and/or dose dispensing after the operational state has been changed. Thus, the interaction between the electronic device and the drug delivery device may not lead to an immediate dispensing of the medicament. The interaction and/or signal exchange between the electronic device and the drug delivery device may take place before a dispensing button or a setting element is operated by the user. In at least one embodiment, the method performable with an electronic device in order to communicate with a drug delivery device comprises determining whether a user of the electronic device is authorized to operate the drug delivery device based on first information and second information, wherein the first information is indicative for a prescription of the drug to a patient and the second information is indicative for the drug the drug delivery device is foreseen to dispense. If the user is authorized to use the drug delivery device, an output signal which is foreseen to be communicated to the drug delivery device in order to enable changing an operational state of the drug delivery device is generated.

Safety of prescription drugs often relies on paper-based prescriptions and the personal handing over of the drug to the person it has been prescribed to. For immobile patients, in telemedicine use cases and/or pandemic circumstances, this is increasingly burdensome, time consuming and poses additional risks. If the drug is distributed by postal delivery, additional efforts have to be made to ensure delivery to the right recipient to ensure e.g. safety for children in the household. After all this, the risk of a drug mix-up still remains unsolved (e.g. mix-up by pharmacist, error in logistics). The present disclosure suggests, inter alia, a solution to this problem, e.g. for injectable drug distributed in disposable needle-based injection system (NIS) pen, auto-injectors or even inhalers. By determining whether the user is authorized to operate a drug delivery device based on first information and second information using an electronic device and only then generating an output signal for enabling a change of an operational state of the drug delivery device, the above-mentioned problems, can, inter alia, be solved.

According to at least one embodiment, the first information is extracted from a first signal transmitted from an external device to the electronic device. The external device may be a further or second electronic device, like a computer or smartphone. Particularly, the external device is different from the electronic device for performing the method and also different from the drug delivery device.

According to at least one embodiment, the second information is extracted from a second signal transmitted from the drug delivery device to the electronic device.

The method may also comprise receiving of the first and/or second signal. For receiving the first and/or the second signal, the electronic device may comprise one or more communication components or communication interfaces, respectively.

The method may comprise a step of generating and/or sending a first request signal foreseen to be transmitted to the external device, e.g. via a cloud service, in order to request the first signal. Thus, the electronic device may be configured to generate and/or send a first request signal. In response to the first request signal, the first signal may be transmitted to the electronic device. By way of example, for generating and sending the first request signal, a code, like a QR code, on the drug delivery device or a package of the drug delivery device is scanned, e.g. with the electronic device. The code may be indicative for the drug of the drug delivery device or the drug the drug delivery device is foreseen to dispense. In response to scanned code, the first request signal may be generated and sent for requesting a prescription of the drug extracted from the code. The step of generating and/or sending the first request signal is particularly performed before the step of determining whether the user of the electronic device is authorized to operate the drug delivery device.

The method may comprise a step of generating and/or sending a second request signal to be transmitted to the drug delivery device in order to request the second signal. Thus, the electronic device may be configured to generate and/or send a second request signal. In response to the request signal, the second signal may be generated. For example, the drug delivery device generates, in response to the second request signal, the second signal and sends it to the electronic device or the drug delivery device influences the second request signal such that it is transformed to the second signal and then received by the electronic device. The step of generating and/or sending the second request signal is particularly performed before the step of determining whether the user of the electronic device is authorized to operate the drug delivery device.

The communication component(s)/interface(s) of the electronic device may also be configured to generate and/or send the request signals.

According to at least one embodiment, the first signal and/or the second signal are wirelessly transmitted signals. The communication component(s) or the communication interface(s) of the electronic device, respectively, may therefore be wireless communication component(s) or wireless communication interface(s).

According to at least one embodiment, the second signal is an RFID signal. The drug delivery device may comprise an RFID tag, e.g. a passive or an active RFID tag. The electronic device may comprise an RFID reading device, e.g. an active or passive reading device. For example, the second signal is a Near Field Communication (NFC) signal. A communication component of the electronic device may then be an NFC component.

Alternatively, the second signal may be a Bluetooth signal. Accordingly, the electronic device and the drug delivery device may each comprise a Bluetooth communication component.

According to at least one embodiment, the first signal is a Long Range Communication signal, particularly a mobile communication signal. Additionally or alternatively, the first signal is transmitted by a cloud service. The first signal may be transmitted via Wi-Fi, LTE, 3G, 4G, 5G or any other mobile communication standard.

For example, a doctor or healthcare professional (HCP) issues the prescription of the drug to the patient, this prescription is transmitted in digital form wirelessly from a device of the doctor or HCP to a cloud service and the first signal with the information about the prescription stored therein is then transmitted to the electronic device.

According to at least one embodiment, the first and/or the second signal are cryptographically secured, e.g. via a public-key system or an asymmetric crypto system using private and public keys, respectively. The first and/or second signal may, therefore, be tamper-proof.

According to at least one embodiment, extracting the first and/or the second information comprises decrypting the first and/or the second signal. Thus, the electronic device may be configured to decrypt the first and/or the second signal.

According to at least one embodiment, the first and/or the second signal are authenticated, particularly in order to check the origin of the first and/or second signal. This may be done before generating the output signal. For example, only if the origin of the first and/or second signal is trusted or correct, the output signal is generated. Thus, the electronic device may be configured to authenticate the first and/or second signal.

According to at least one embodiment, the method comprises generating a power signal in order to transfer energy to the drug delivery device, e.g. via induction. For example, the power signal is configured to be wirelessly transmitted to the drug delivery device in order to provide the drug delivery device with electric energy. Thus, the electronic device may serve as an energy source for a locking mechanism and/or an actuator element of the drug delivery device, wherein the energy transmission may be via induction.

For example, the RFID component or the Near Filed Communication component is used to transfer energy from the electronic device to the drug delivery device. The drug delivery device may comprise a pre-charge capacitor for this purpose. The second signal may comprise the power signal.

Additionally or alternatively, a charging system, e.g. a smartphone reverse charging system, like a QI system, is used to transfer energy from the electronic device to the drug delivery device.

It is also possible that the drug delivery device comprises an own power source, e.g. a battery, for providing the drug delivery device with electric energy. The energy of the battery can be used alone or in combination with the transmitted energy to operated the drug delivery device.

According to at least one embodiment, the change of the operational state of the drug delivery device is associated with a mechanical change in a mechanism unit of the drug delivery device. For example, the change of the operational state is associated with a movement of an element of the mechanism unit.

According to at least one embodiment, the change of the operational state is a change between a state where setting a drug dose and/or dispensing a drug dose is prevented and a state where setting a drug dose and/or dispensing a drug dose is enabled.

According to at least one embodiment, the output signal is generated without a communication connection to the external device or the cloud service. For example, the output signal is generated, when the electronic device is offline, e.g. not wirelessly connected to the internet. By way of example, the first signal may be received, then the connection to the external device or the cloud service may be interrupted and then the second signal is received and/or the output signal is generated.

For example, the prescription of the first information is valid for a predetermined time, like several hours or several days. If the output signal generatable based on this prescription is not generated within the predetermined time, the prescription becomes invalid and an output signal can no longer be generated based on this prescription. A new first signal may then be required.

According to at least one embodiment, the first information is indicative for a plurality of prescriptions of drugs to a user. Each prescription may be uniquely assigned to one drug delivery device. The first information may be assigned to a multi-pack of drug delivery devices. For example, in order to receive the respective first signal, the user of the electronic device has to first scan a code, like a QR code, on the multi-pack, then a first request signal is sent to external device and/or the cloud service and in response to the first request signal, the first signal comprising the information about the plurality of receipts is generated and sent to the electronic device. The first information of the first signal in order to generate the output signal may then be used offline.

Each prescription may be assigned a time window. For example, the output signal assigned to one prescription can only be generated within this time window. This may avoid an overdose, particularly if the time windows assigned to different prescriptions do not overlap or do not completely overlap.

The method described herein has, inter alia, the following benefits:
Drug mix-up prevention: User can only unlock the correct type of drug delivery device.
Safe distribution: Personal delivery to patient is no longer required, as only the patient can use the drug. Depending on markets, the drug could be made commonly available even outside of pharmacies.
Safe postal delivery: No personal delivery required. Parcels could be safely delivered to children even, as they cannot use the drug delivery device.
Supports telemedicine: Prescriptions can be deployed by HCP remotely via cloud service within live video sessions—no personal contact or paper-based prescriptions required.
Wide compatibility: The concept may use technology like NFC and would be compatible with common smartphones.
Emergency medication: Patients could carry a (dangerous) emergency injector/inhaler (e.g. epi-pen) but require to call up their health center to assess the situation and give ad-hoc permission (by a transmitted prescription) to use it.
Re-use protection: Transmitted prescriptions may be invalidated when used by cryptographic means and cannot be copied or reused.

Next, the electronic device is specified. The electronic device is configured to perform the method described herein. Therefore, all features described in connection with the method are also disclosed for the electronic device and vice versa.

According to at least one embodiment, the electronic device comprises at least one processor. The at least one processor may particularly be configured to extract the first and/or second information from the first and/or second signal and/or to decrypt the first and/or the second signal and/or to determine whether the user is authorized to operate the drug delivery device and/or to generate the output signal. The electronic device may be a handheld device and/or wearable device. Particularly, the electronic device may be a tablet PC, a smartphone or a smartwatch.

According to at least one embodiment, the electronic device comprises one or more communication components configured to receive signals and/or to send an output signal. For example, the communication component(s) is (are) configured to receive wireless signals and/or to send a wireless output signal. Thus, the communication component(s) may be wireless communication component(s).

According to at least one embodiment, the electronic device comprises an RFID component, particularly an NFC component. The communication component may be a reading device for reading and RFID tag or an NFC tag.

According to at least one embodiment, the electronic device comprises at least one Long Range Communication component, particularly a mobile communication component. For example, the electronic device comprises a Wi-Fi-, LTE-, 3G-, 4G- and/or 5G-communication component.

According to at least one embodiment, the electronic device comprises an inductive charging component, e.g. a coil.

Next, the computer program and the computer-readable data carrier are specified. The computer program and the computer-readable data carrier comprise instructions which, when executed by the electronic device, cause the electronic device to carry out the method described herein. The computer program may be an APP.

Next, the drug delivery device is specified. The drug delivery device may, in particular, be the drug delivery device with which the electronic device communicates when performing the method. Therefore, all features disclosed in connection with the method are also disclosed for the drug delivery device and vice versa.

The drug delivery device specified herein may be an injection device, e.g. a needle-based injection device, or an inhaler. The drug delivery device may be an autoinjector and/or a variable dose device or a fixed dose device and/or a pen type device, e.g. a dial extension pen. The drug delivery device may be a disposable device.

According to at least one embodiment, the drug delivery device comprises a mechanism unit. The mechanism unit may comprise a dispense mechanism for dispensing a drug dose and/or a setting mechanism for setting a drug dose.

The dispense mechanism and/or the setting mechanism may comprise several elements which interact with each other during dose dispensing or dose setting. For example, a coupling between two or more elements of the mechanism unit is changed when switching from dose setting to dose dispensing or vice versa. For example, two or more elements are splined during dose setting such that they are rotationally fixed to each other, wherein the splined coupling is released for dose dispensing so that these elements rotate relative to each other during dose dispensing.

For example, the dispense mechanism comprises a plunger rod configured to act on a drug reservoir in order to dispense a drug dose. The mechanism unit may be configured such that the plunger rod moves axially in distal direction during dose dispensing. The plunger rod may also rotate during dose dispensing, e.g. due to a threaded engagement with a further element of the mechanism unit, like a drive element. For example, during setting a drug dose, the plunger rod does not move.

The dispense mechanism may also comprise an energy member in order to provide energy for dispensing a drug dose. The energy member may provide energy for moving the plunger rod in distal direction. For example, the energy member is a drive spring, like a compression spring or a torsion spring, or a gas cartridge or an electric motor. Alternatively, no additional energy member for moving the plunger rod is used. The force needed for moving the plunger rod and dispensing a drug dose may then have to be provided by the user.

The dispense mechanism may comprise a drive element, e.g. a drive sleeve. The drive sleeve may circumferentially surround the plunger rod. The drive element may be threadedly engaged with the plunger rod. During dispensing a drug dose, the drive element may move in distal direction, e.g. without rotation, and may thereby force the plunger rod to rotate and also move in distal direction.

The setting mechanism may comprise a setting element, e.g. a dial sleeve and/or a number sleeve. During dose setting, the drive element may be splined to the setting element. For example, during dose setting, the drive element and the setting element move together, e.g. on a helical path, in a proximal direction but may not move relative to each other. During dose dispensing, the splined coupling between the drive element and the setting element may be released. For example, during dose dispensing, the setting element moves back on the helical path in distal direction but the drive element only moves axially in distal direction without rotation. For realizing the splined coupling and for releasing the splined coupling between the drive element and the setting element, the mechanism unit may comprise a clutch and/or a clicker arrangement and/or a clutch spring.

The mechanism unit may comprise a user interface member configured to be operated by a user, e.g. to be touched by a user, in order to dispense a drug dose. For example, the user interface member is a button or a knob. For example, in order to dispense a drug dose, the user interface member has to be pushed in a distal direction by the user. This user interface member may also be referred to as dose dispense member.

The mechanism unit may also comprise a user interface member configured to be operated by a user, e.g. to be touched by a user, in order to set a drug dose. For example, for setting a drug dose, the user has to rotate and/or move the user interface member in a proximal direction. This user interface member may also be referred to as dose setting member.

The user interface member for setting a drug dose may be at the same time the user interface member for dispensing a drug dose.

According to at least one embodiment, the mechanism unit comprises a housing element. The housing element may be a sleeve. For example, the housing element circumferentially surrounds other or all of the elements of the mechanism unit. The housing element may comprise an outer surface which forms an outer surface of the drug delivery device which can be touched by a user.

For the present description, if not stated otherwise, movement of a member or element or feature of the drug delivery device particularly means a movement relative to the housing element.

The drug delivery device specified herein may be elongated and/or may comprise a longitudinal axis, e.g. a main extension axis. Additionally or alternatively, the drug delivery device may have a rotational symmetry with respect to the longitudinal axis. A direction parallel to the longitudinal axis is herein called an axial direction. By way of example, the drug delivery device may be cylindrically-shaped.

Furthermore, the drug delivery device may comprise an end, e.g. a longitudinal end, which may be provided to face or to be pressed against a skin region of a human body. This end is herein called the distal end. A drug or medicament may be supplied via the distal end. The opposing end is herein called the proximal end. The proximal end is, during usage, remote from the skin region. The axial direction pointing from the proximal end to the distal end is herein called distal direction. The axial direction pointing from the distal end to the proximal end is herein called proximal direction. A distal end of a member or element or feature of the drug delivery device is herein understood to be the end of the member/element/feature located most distally. Accordingly, the proximal end of a member or element or feature is herein understood to be the end of the element/member/feature located most proximally.

In other words, distally is used herein to specify directions, ends or surfaces which are arranged or are to be arranged to face or point towards a dispensing end of the drug delivery device or components thereof and/or point away from, are to be arranged to face away from or face away from the proximal end. On the other hand, proximal is herein used to specify directions, ends or surfaces which are arranged or are to be arranged to face away from or point away from the dispensing end and/or from the distal end of the drug delivery device or components thereof. The distal end may be the end closest to the dispensing end and/or furthest away from the proximal end and the proximal end may be the end furthest away from the dispensing end. A proximal surface may face away from the distal end and/or towards the proximal end and a distal surface may face towards the distal end and/or away from the proximal end. The dispensing end may be a needle end where a needle unit is or is to be mounted to the device, for example.

A direction perpendicular to the longitudinal axis and/or intersecting with the longitudinal axis is herein called radial direction. An inward radial direction is a radial direction pointing towards the longitudinal axis. An outward radial direction is a radial direction pointing away from the longitudinal axis. The term “angular direction”, “azimuthal direction” or “rotational direction” are herein used as synonyms. Such a direction is a direction perpendicular to the longitudinal axis and perpendicular to the radial direction.

According to at least one embodiment, the mechanism unit is configured to be operatively coupled with a drug reservoir unit.

The drug reservoir unit may comprise or may be a drug reservoir and/or a drug reservoir holder for holding a drug reservoir. The drug reservoir holder may be configured to hold the drug reservoir such that the drug reservoir is not movable relative to the drug reservoir holder. The drug reservoir may be a cartridge connectable with an injection needle or may be a syringe comprising an injection needle. The drug reservoir may comprise a drug, e.g. several doses of a drug.

The drug reservoir may have a distal end for dispensing the drug. The distal end may be the end comprising a needle or the end to be connected with a needle. The drug reservoir may comprise a stopper sealing the drug reservoir in proximal direction.

“Operatively coupled” particularly means that the mechanism unit and the drug reservoir unit are mechanically coupled or connected, respectively, particularly releasable coupled or connected. For this purpose, the mechanism unit may comprise an interface feature for forming a connection interface connecting the mechanism unit to the drug reservoir unit. The interface feature may comprise a thread configured to engage into a thread of the drug reservoir unit for forming the connection interface. Alternatively, the interface feature may be configured to establish a snap connection to the drug reservoir unit. When coupled, the drug reservoir unit may be fixed with respect to the housing element so that, e.g., the drug reservoir unit is not movable with respect to the housing element in axial direction. Additionally or alternatively, “operatively coupled” may mean that the mechanism unit and the drug reservoir unit are coupled to exchange information, e.g. electrical signals or currents.

According to at least one embodiment, the mechanism unit is configured to enable a dispense process for dispensing a drug dose, e.g. a set drug dose. Particularly, the mechanism unit may be configured to act on a drug reservoir, particularly a drug reservoir of the drug reservoir unit, during the dispense process. When the mechanism unit acts on the drug reservoir, the mechanism unit may push the stopper in distal direction in order to dispense a drug dose. For example, the plunger rod of the mechanism unit thereby abuts against the stopper and pushes the stopper in distal direction. Performing the dispense process may require the user to operate the dose dispense member.

According to at least one embodiment, the mechanism unit comprises an arrangement for changing an operational state of the mechanism unit, particularly, for changing from a first operational state into a second operational state or vice versa. The second operational state is, in particular, a state in which at least one functionality of the mechanism unit is enabled or performed which is disabled or not performed in the first operational state.

The arrangement for changing the operational state may comprise one or more components which interact with each other. For example, the arrangement comprises mechanical and/or electrical components. By way of example, the arrangement comprises one or more of: an electromechanical actuator, a control unit, a display, an energy source. The control unit may comprise a processor, e.g. an IC-chip. The control unit may be a micro controller. The energy source may be a battery.

According to at least one embodiment, the mechanism unit comprises a communication component for receiving an output signal transmitted from an electronic device to the drug delivery device. The communication component may be a wireless communication component. The communication component may be a Bluetooth component or an RFID component or an NFC component. The communication component may comprise an RFID tag or NFC tag.

According to at least one embodiment, the communication component is configured to establish a communication connection or chain with the electronic device, e.g. an encrypted and/or secured communication connection or chain. The communication component of the drug delivery device may be configured to send a second signal to the electronic device comprising information about the drug delivery device, particularly about a drug the drug delivery device comprises or is configured to dispense. The drug delivery device may be configured to identify itself, e.g. in response to a received request signal.

According to at least one embodiment, the mechanism unit is configured such that operation of the arrangement in order to change the operational state of the mechanism unit is prevented unless an output signal from the electronic device is received via the communication component.

For example, the mechanism unit is configured to enable operation of the arrangement for changing the operational state, particularly to only enable it, when the output signal is received. The operation of the arrangement changing the operational state may happen automatically when the output signal is received. Alternatively, operation of the arrangement may additionally require a further process to be performed, like a manual operation by a user of the drug delivery device. Particularly, receiving the output signal may be a precondition for the operation of the arrangement.

The mechanism unit may be configured to determine whether the output signal is received and to prevent and/or only enable operation of the arrangement unless or when it is determined that the output signal is received, respectively. For example, if the output signal is not received, changing the operational state is prevented.

The mechanism unit may also be configured to authenticate and/or to decrypt the output signal, particularly in order to check the origin of the output signal. This may be done before enabling the operation of the arrangement. For example, only if the origin of the output signal is trusted or correct, the mechanism unit enables of the arrangement.

According to at least one embodiment, the drug delivery device is a device for self-administration. Particularly, using the drug delivery device can be done by a patient without professional support.

According to at least one embodiment, the arrangement comprises an electromechanical actuator. The electromechanical actuator may comprise an electric motor and/or an electromagnet.

According to at least one embodiment, operation of the arrangement for changing the operational state comprises operation of the actuator. The actuator may be configured such that, when operated, it moves an actuator element of the actuator between a first position and a second position.

An electromechanical actuator is herein understood to be an actuator which converts an electrical signal into a movement of an actuator element. For example, when operated, the actuator may move the actuator element from the first position into the second position and/or vice versa. The movement between the first position and the second position may be a movement in axial and/or rotational and/or radial direction.

The mechanism unit, particularly the arrangement, may comprise a control unit for operating the actuator. For example, for operating the actuator, the control unit sends an electrical signal. The control unit may be configured to only operate the actuator when the output signal is received. When no output signal is received, the actuator may not be operated or may not be operable. According to at least one embodiment, the drug delivery device comprises a drug reservoir unit comprising a drug and coupled to the mechanism unit.

Furthermore, a set is specified. The set may comprise an electronic component as specified herein and a drug delivery device as specified herein.

Hereinafter, the method, the electronic device and the drug delivery device will be explained in more detail with reference to drawings on the basis of exemplary embodiments. Same reference signs indicate similar, similar acting or same elements in the individual figures. However, the size ratios involved are not necessarily to scale, individual elements may rather be illustrated with exaggerated size for better understanding.

### BRIEF DESCRIPTION OF THE FIGURES

FIG. 1 shows a schematic illustration of an exemplary embodiment of the method, an exemplary embodiment of the electronic device and a first exemplary embodiment of the drug delivery device,

FIGS. 2 to 6 show a second exemplary embodiment of a drug delivery device in different views,

FIGS. 7 to 12 show a third exemplary embodiment of a drug delivery device in different views,

FIGS. 13 and 14 show a fourth exemplary embodiment of the drug delivery device in different views.

### DETAILED DESCRIPTION

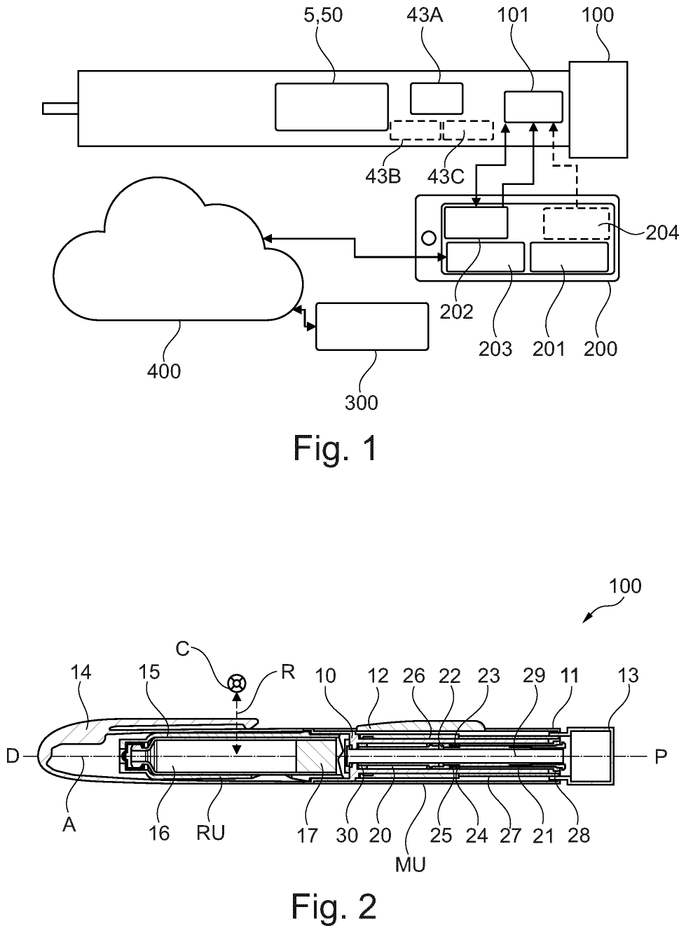

FIG. 1 shows a schematic illustration of an exemplary embodiment of the method. The method is performed with an electronic device 200. The electronic device 200 is, e.g., a smartphone. The electronic device 200 comprises a processor 201, communication components 202, 203 and an inductive charging component 204.

The method comprises a step in which it is determined, based on first information and second information, whether a user of the electronic device 200 is authorized to operate a drug delivery device 100. The method further comprises a step in which an output signal is generated if the user is authorized to use the drug delivery device 100. The output signal is foreseen to be communicated to the drug delivery device 100 in order to enable changing an operational state of the drug delivery device 100. These two steps may be performed by the processor 201.

The first information and the second information may be extracted from a first signal and a second signal, e.g. using the processor 201. The first information is indicative for a prescription of the drug to a patient and the second information is indicative for the drug the drug delivery device 100 is foreseen to dispense. For receiving the first signal and the second signal, the communication components 202 and 203 may be used.

Determining whether the user of the electronic device 200 is authorized to operate the drug delivery device 100 may be done by comparing the identity of the user of the electronic device 200 with the identity of the patient the prescription is for and additionally comparing the drug the drug delivery device 100 is foreseen to dispense to the drug of the prescription. If the user and the patient are identical and if the drug of the prescription and the drug of the drug delivery device 100 are identical, it is determined that the user is indeed authorized to operate the drug delivery device 100. In order to provide its identity to the electronic device 200, the user may first have to enter a password and/or provide a fingerprint and/or to a face scan.

The communication component 203 may be a Long Range Communication component, like in Wi-Fi-, LTE-, 3G-, 4G- or 5G-communication component configured to receive the first signal. The first signal may be wirelessly transmitted from an external device 300 via a cloud service 400 to the electronic device 200. The external device 300 may be assigned to a doctor or healthcare professional which prescribes the drug to the patient. This prescription or the information about this prescription, respectively, is then stored in the first signal. The first signal may be an encrypted signal and decrypting of the signal may done by the processor 201 of the electronic device 200.

The method may also comprise generating and sending a first request signal in order to request the prescription. This first request signal may be generated and/or sent by the communication component 203. The request signal may then be forwarded via the cloud service 400 to the external device 300.

The communication component 202 may be configured to communicate with the drug delivery device 100. For example, the communication component 202 comprises an RFID communication component, like a Near Field Communication (NFC) component. The communication component 202 may be configured to send a second request signal and to receive the second signal. The second request signal and the second signal may be wirelessly transmitted signals. In order to receive the second signal, the user of the external device 200 may bring the external device 200 close to the drug delivery device 100 which may then send the second request signal to the drug delivery device 100 and may accordingly receive the second signal in which information about the drug delivery device 100, particularly the drug of the drug delivery device 100 or the drug which the drug delivery device 100 is foreseen to dispense, is stored.

The communication component 202 may further be configured to send the output signal generated if it was determined that the user is authorized to operate the drug delivery device 100.

The inductive charging component 204 may be configured to provide the drug delivery device 100 with electric energy, particularly via induction.

The drug delivery device 100 may be an injection device, e.g. an autoinjector and/or a variable dose device. The drug delivery device 100 comprises a communication component 101. The communication component 101 may comprise an RFID communication component, like an NFC component. The communication component 101 may comprise an RFID tag. The communication component 101 is, in particular, configured to receive the output signal of the electronic device 200 and/or to influence the second request signal of the electronic device 200 and/or to send the second signal to the electronic device 200.

The drug delivery device 100 is configured to enable a change of its operational state when the output signal is received. For this purpose, the drug delivery device 100 comprises an arrangement which, in the present case, comprises an electromechanical actuator 5, a control unit 43A and the battery 43B. The actuator 5 comprises an actuator element 50 which is moved when the actuator 5 is operated. The battery 43B may be omitted if the electric energy transmitted to the drug delivery device 100 from the external device 200 is sufficient to operate the control unit 43A and/or the electromechanical actuator 5. The drug delivery device 100 comprises a pre-charge capacitor 43C for storing energy transmitted from the external device 200.

The control unit 43A is configured to operate the actuator 5 in order to change the operational state of the drug delivery device 100. For example, the change of the operational state of the drug delivery device 100 is a change from a state where a dispense process in order to dispense a drug dose and/or a setting process in order to set a drug dose is prevented to a state in which such a process or such processes are enabled. The communication component 101 may be configured to forward the received output signal to the control unit 43A. The control unit 43A is, e.g., configured to only operate the actuator 5 for changing the operational state of the drug delivery device 100 when receiving the forwarded output signal. Otherwise, the actuator 5 is not operated or not operable. Details about the construction of the arrangement, particularly of the actuator 5, are explained further below.

FIG. 2 shows a second exemplary embodiment of a drug delivery device 100 in a cross-sectional view. The drug delivery device 100 is a variable dose device, in which different doses of a drug to be dispensed can be set or dialed, respectively, by a user. The drug delivery device 100 is a dial extension pen.

FIG. 2 also indicates the coordinate system used herein for specifying positions of members or elements or features of the drug delivery device 100. The distal direction D and proximal direction P run parallel to the longitudinal axis A. The longitudinal axis A is a main extension axis of the device 100. The radial direction R is a direction perpendicular to the longitudinal axis A and intersecting with the longitudinal axis A. The azimuthal direction C, also referred to as angular direction or rotational direction, is a direction perpendicular to the radial direction R and to the longitudinal axis A. The different directions and axes will not be indicated in the following figures in order to increase the clarity of the figures.

The drug delivery device 100 comprises a mechanism unit MU with a setting mechanism and a dispense mechanism. The setting mechanism is configured for setting a drug dose and the dispense mechanism is configured for dispensing a drug dose. The functional principles of the mechanisms are explained further below.

The mechanism unit MU comprises an inner body 10 and a housing element 11, in the following also referred to as outer body 11. The inner body 10 and the outer body 11 are fixedly connected to each other, i.e. they cannot be rotated or moved axially with respect to each other. The outer body 11 forms an outer surface of the drug delivery device 100 which can be touched or grabbed by a user.

The drug delivery device 100 further comprises a cap 14 and a user interface member 13 in form of a knob 13. The knob 13 is a dose setting member configured to be operated by a user for setting a drug dose. At the same time, the knob 13 is dose dispense member configured to be operated by a user in order to dispense a drug dose.

A drug reservoir unit RU comprising a reservoir 16 and a reservoir holder 15 is received within the cap 14. A drug is filled into the reservoir 16. The reservoir 16 is sealed in proximal direction P by a stopper 17.

The drug reservoir unit RU is operatively coupled or connected, respectively, to the mechanism unit MU. The mechanism unit MU is configured to enable a dispense process for dispensing a drug dose by acting on the drug reservoir 16. For dispensing a drug dose, the stopper 17 is pushed in distal direction D by a plunger rod 29 of the mechanism unit MU. The coupling between the mechanism unit MU and the reservoir unit RU is realized by the inner body 10 being coupled to the reservoir holder 15 via a connection interface which might be a snap connection or a threaded connection. The coupling is preferably reversible. For example, the drug reservoir unit RU is axially and rotationally fixed to the inner body 10 by the coupling.

The mechanism unit MU further comprises a number sleeve 26 and a dial sleeve 27 which are fixedly coupled to each other (e.g. they cannot rotate or move axially relative to each other). The dial sleeve 27 may comprise an inner thread which is engaged with an outer thread of the inner body 10. On an outer surface of the number sleeve 26, numbers may be shown. The user can see the numbers through a window 12 of the mechanism unit MU. The window 12 may comprise a lens. The window 12 is formed in the outer body 11. The numbers visible in the window 12 indicate to a user the set/dialed dose. Due to the threaded coupling between the dial sleeve 27 and the inner body 10, the dial sleeve 27 and the number sleeve 26 are moved on a helical path in proximal direction relative to the body 10, 11 during setting a drug dose and dispensing a drug dose as will be explained further below.

The mechanism unit MU also comprises a drive sleeve. The drive sleeve comprises a distal drive sleeve 20, a proximal drive sleeve 21 and a drive sleeve coupler 22 coupling the distal drive sleeve 20 to the proximal drive sleeve 21. For setting a drug dose and dispensing a drug dose, the distal drive sleeve 20 and the proximal drive sleeve 21 are fixedly coupled to each other via the drive sleeve coupler 22 so that these elements can neither rotated nor move axially relative to each other. The distal drive sleeve 20 may comprise an inner thread which is engaged with an outer thread of the plunger rod 29. An outer thread of the distal drive sleeve 20 may be engaged to an inner thread of a last dose nut 30, the function of which will be explained further below.

Furthermore, the mechanism unit MU comprises a clutch 28, which is fixedly coupled to the knob 13 so that, during setting a drug dose and dispensing a drug dose, the clutch 28 and the knob 13 are not rotated or moved axially relative to each other. The clutch 28 is coupled to the proximal drive sleeve 20 via a splined engagement. This splined engagement may allow a certain axial movement of the clutch 28 relative to the proximal drive sleeve 21 but does not allow a relative rotation between these two elements.

A distal clicker 23, a proximal clicker 24 and a clutch spring 25 of the mechanism unit MU are arranged between the clutch 28 and the drive sleeve coupler 22. The clutch spring 25 is coupled to the drive sleeve coupler 22 and to the distal clicker 23. The distal clicker 23 is configured to abut against the proximal clicker 24 in proximal direction P. The proximal clicker 24 is configured to abut against the clutch 28 in proximal direction P. Thus, the clutch spring 25 is configured to bias the distal clicker 23, the proximal clicker 24 and the clutch 28 in proximal direction P relative to the drive sleeve coupler 22 (see also FIGS. 15 and 17 for a more detailed view).

The distal clicker 23 may be permanently splined to the proximal drive sleeve 21 so that a relative rotation between these two elements is prevented. However, a certain axial movement between the distal clicker 23 and the proximal drive sleeve 21 may be allowed. The proximal clicker 24 may be permanently splined to the inner body 10 so that a relative rotation between these two elements is prevented, whereas a certain relative axial movement may be allowed.

The distal face of the clutch 28 and the proximal face of the proximal clicker 24 may both be toothed so that these two faces may engage into each other. Furthermore, the distal face of the proximal clicker 24 and the proximal face of the distal clicker 23 may both be toothed so that these two toothed faces can engage into each other. A proximal face of the clutch 28 may be toothed, e.g. dog toothed, and may be arranged to engage a toothed, e.g. dog toothed, distal face of the dial sleeve 27.

FIG. 2 shows the drug delivery device 100 when no dose is set (0 units/0 unit position). Dose setting may be allowed in discrete units of 1, e.g. from 0 to 80 units. For setting a desired drug dose, the user has to rotate the knob 13. This is done without pressing on the knob 13 in distal direction D. As long as one does not press on the knob 13 in distal direction D, a dog toothed engagement between the clutch 23 and the drive sleeve 27 is established due to the clutch spring 25 either biasing the clutch 28 in proximal direction P or at least preventing the clutch 28 from moving in distal direction D on its own. The dog toothed engagement between the clutch 28 and the dial sleeve 27 has as a consequence that the two elements are rotationally locked to each other so that, when the knob 13 is rotated, also the dial sleeve 27 and the number sleeve 26 are rotated. Since the dial sleeve 27 is threadedly engaged with the inner body 10, rotating the knob 13 has as a consequence that the knob 13, the clutch 28, the dial sleeve 27 and the number sleeve 26 move on a helical path in proximal direction P relative to the body 10, 11. Thereby, the numbers of the number sleeve 26 visible through the window 12 increase.

Since the proximal drive sleeve 21 is splined to the clutch 28, also the proximal drive sleeve 21 and with it the distal drive sleeve 20 and the drive sleeve coupler 22 are moved on the helical path in proximal direction P relative to the inner body 10.

The plunger rod 29 comprises two outer threads with opposite hand which overlap with each other. The plunger rod 29 is threadedly engaged with the inner thread of the distal drive sleeve 20. The threads are chosen such that during the helical movement of the distal drive sleeve 20 in proximal direction P, the plunger rod 29 does not rotate and is also not moved axially.

The last dose nut 30 may be splined to the inner body 10 and, therefore, cannot rotate relative to the inner body 10. Due to the threaded engagement of the last dose nut 30 with the distal drive sleeve 20, the last dose nut 30 is forced to move in proximal direction P during setting a drug dose. When the maximum dose has been set (e.g. 80 units—independently of whether it has been set in only one drug setting process or several drug setting processes), the last dose nut 30 establishes a rotation-lock interface with the distal drive sleeve 20 so that the last dose nut 30 can no longer rotate relative to the distal drive sleeve 20. As a consequence of this, the distal drive sleeve 20 can no longer be rotated and no further drug dose can be set. The drug delivery device 100 then as to be reset to its initial state.

During setting a drug dose, the toothed faces of the distal clicker 23 and the proximal clicker 24 facing each other ratchet over each other thereby creating a click sound which indicates to a user that a drug dose is set. For this purpose, the teeth of the two faces are preferably formed as shallow triangles so that relative rotation between the clickers 23 and 24 is possible leading to a repeated slight compression and decompression of the clutch spring 25.

After the desired dose has been set, the user can now press on the knob 13 in distal direction D in order to dispense the set drug dose. Thereby, the distally directed force on the knob 13 is transferred from the knob 13 via the clutch 28 to the proximal clicker 24, from there to the distal clicker 23 and this compresses the clutch spring 25. The two clickers 23 and 24 are now pressed against each other and their toothed faces are engaged. Relative rotation between the two clickers 23, 24 is now prevented. Since the proximal clicker 24 is splined to the inner body 10 and the distal clicker 23 is splined to the proximal drive sleeve 21, the proximal drive sleeve 21 can no longer rotate relative to the inner body 10. However, since the proximal drive sleeve 21 is also splined to the clutch 28, also the clutch 28 and the knob 13 can no longer rotate relative to the inner body 10.

The distally directed force applied to the knob 13 has as a consequence that the clutch 28 together with the knob 13 slightly moves in distal direction D relative to the dial sleeve 27 so that the clutch spring 25 is compressed, as already mentioned. The dog toothed engagement between the dial sleeve 27 and the clutch 28 is thereby released so that the dial sleeve 27 is no longer rotationally locked to the clutch 28. Therefore, when the knob 13 is pressed in distal direction D, the dial sleeve 27 together with the number sleeve 26 can still rotate relative to the inner body 10. When the knob 13 is now moved in distal direction D, a stop against the dial sleeve 27 forces the dial sleeve 27 to also move in distal direction D. Due to the threaded engagement of the dial sleeve 27 with the inner body 10, the dial sleeve 27 together with the number sleeve 26 moves on a helical path in distal direction D. Thereby, the numbers of the number sleeve 26 visible in the window 12 decrease.

At the same time, the clutch 28, the clickers 23, 24 and the drive sleeve 20, 21, 22 are forced to move in distal direction D (without rotation). The threaded engagement between the plunger rod 29 and the distal drive sleeve 20 forces the plunger rod 29 to rotate. A further threaded engagement between the plunger rod 29 and an inner thread of the inner body 10 may then force the plunger rod 29 to also move distally in order to push the stopper 17 inside the cartridge 16 in distal direction D for dispensing the set drug dose. Since the distal drive sleeve 20 is not rotate during dispensing, the last dose not 30 moves together with the distal drive sleeve 20 in distal direction D without changing its position relative to the distal drive sleeve 20.

After having dispensed the set drug dose and when the knob 13 has been completely moved back into its initial position, a new drug dose may be set by again rotating the knob 13 on a helical path in proximal direction P. During this, the plunger rod 29 does not change its position. Only when dispensing a dose, the plunger rod 29 is moved in distal direction D.

As explained with respect to FIG. 2, one user interface member in form of a knob 13 is used for setting a drug dose as well as for dispensing the drug dose. However, it is also possible to use separate user interface members for setting and dispensing a drug dose.

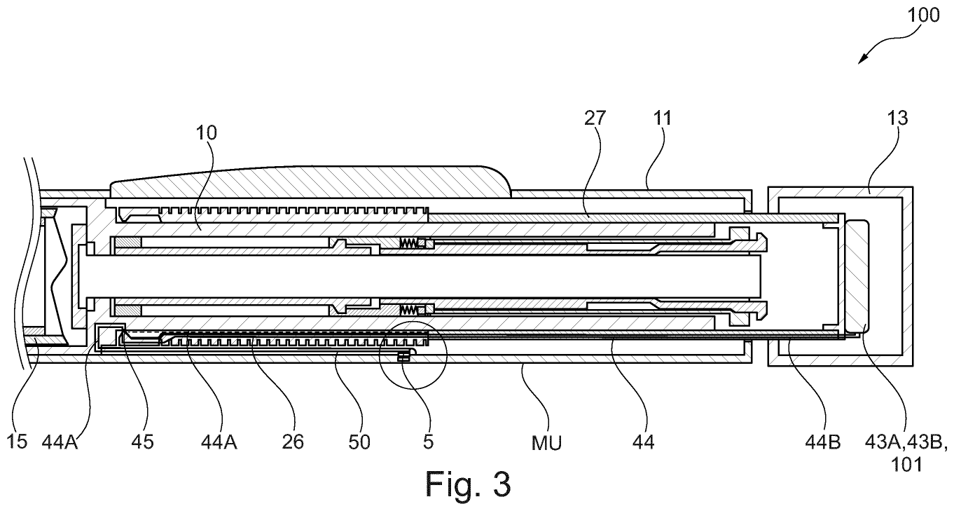

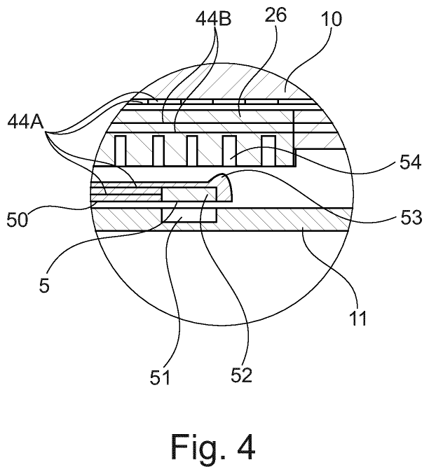

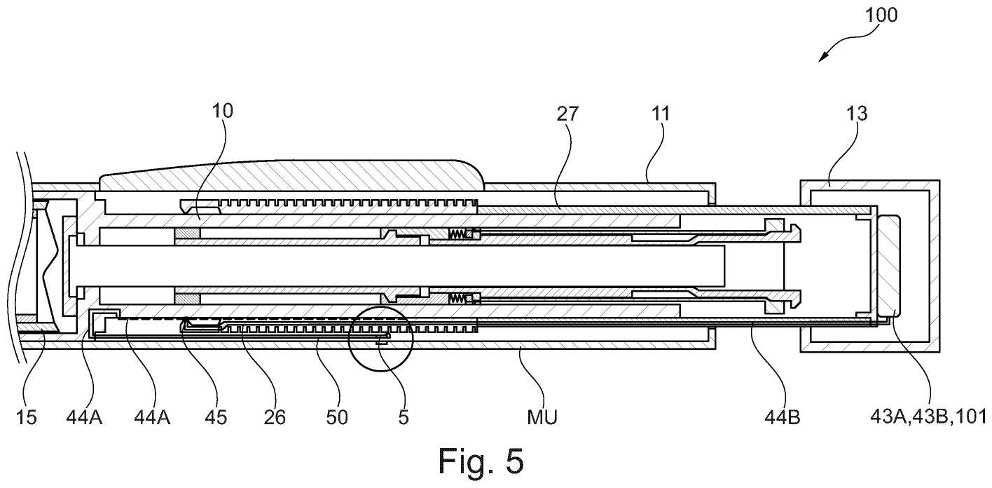

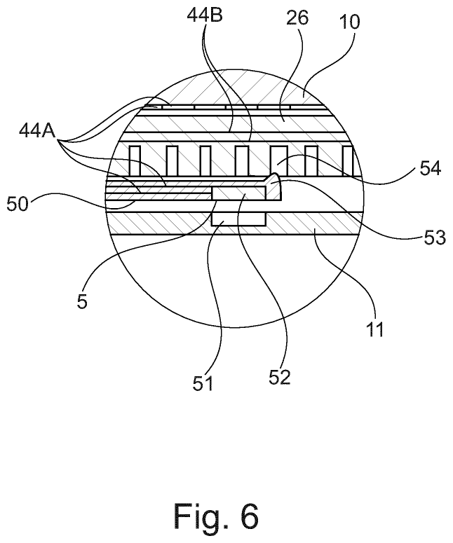

FIGS. 3 to 6 show the drug delivery device 100 of FIG. 2 but in different views than FIG. 2 and with more details. FIGS. 3 and 5 only shows the proximal part of the drug delivery device 100 in order to better illustrate some of the details. FIGS. 4 and 6 shows the circled regions of FIGS. 3 and 5.

As can be seen in FIGS. 3 and 5, the dial sleeve 27 comprises a conductor path 44. The conductor path 44 comprises a winded or helical conductor track, respectively, which is, e.g., arranged at the outer surface of the dial sleeve 27. The pitch of the helical conductor track is preferably the same as the pitch of the helical path on which the dial sleeve 27 moves during setting and dispensing a drug dose.

On the proximal face of the dial sleeve 27, a control system comprising a control unit 43A, a battery 43B and a communication component 101 are arranged. The control unit 43A, the battery 43B and the communication component 101 may be arranged on a PCB mounted on the proximal face of the dial sleeve 27. The control unit 43A may comprise a processor and/or an IC-chip. The control unit 43A and/or the battery 43B may be electrically connected to the conductor path 44. The communication component 101 may be the one described in connection with FIG. 1. It may be electrically connected to the control unit 43A.

As can be seen in FIGS. 3 and 5, the conductor path 44 actually comprises two sections 44A and 44B. These two sections 44A, 44B are assigned to different elements of the drug delivery device 100. The first section 44A is assigned to the body 10, 11 and is fixed to the body 10, 11. The second section 44B is assigned to the dial sleeve 27 and is fixed to the dial sleeve 27 so that it always follows a movement of the dial sleeve 27. Therefore, the two sections 44A, 44B move relative to each other during setting a drug dose and dispensing a drug dose.

In order to always maintain an electrical connection between the first section 44A and the second section 44B during dose setting and dose dispensing, the first section 44A comprises the helical conductor track and a sliding contact 45 is realized between the two sections 44A, 44B. The helical conductor track of the first section 44A assigned to the dial sleeve 27 and having the same pitch as the helical path on which the dial sleeve 27 moves relative to the body 10, 11 during dose setting and dose dispensing in combination with the sliding contact 45 ensures that the two sections 44A, 44B always stay electrically connected during dose setting and dose dispensing.

As can be seen in FIGS. 3 and 5, and in more details in FIGS. 4 and 6, the mechanism unit MU also comprises an electromechanical actuator 5 with an actuator element 50. The actuator element 50 is a displaceable or movable element 50 in form of a flexible arm 50. At one longitudinal end, the flexible arm 50 is fixed to the inner body 10 and the other longitudinal end of the arm 50 is a free end which can be displaced in radial direction R. The arm 50 is orientated in axial direction.

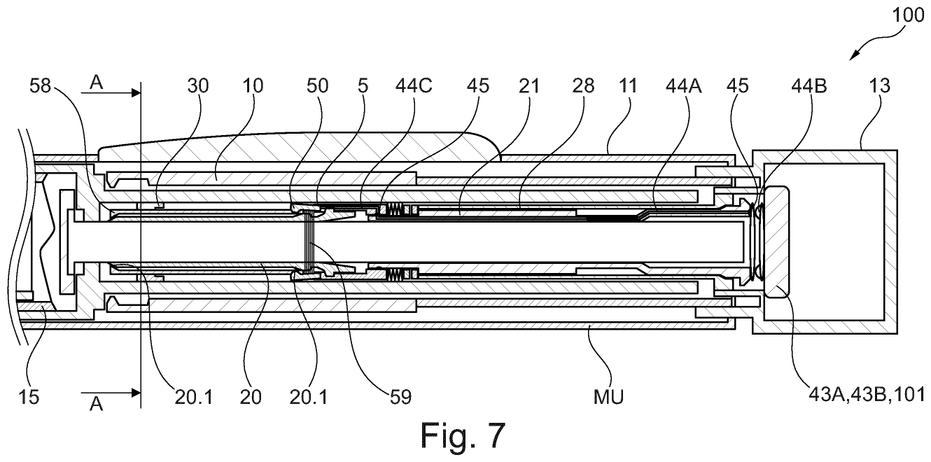

At its free longitudinal end, the arm 50 comprises an electromagnet 52 (see the detailed view of FIG. 7 illustrating the circled region of FIG. 6 in more detail). The electromagnet 52 is configured to change its magnetization when the actuator 5 is operated. The electromagnet 52 is also configured to interact with a magnet 51 in the outer body 11. The magnet 51 axially and/or rotationally overlaps with the electromagnet 52. By changing a current through the electromagnet 52, its magnetization is changed and the arm 50 can be moved between a first and a second position.

FIGS. 3 and 4 show the case where the arm 50 is in the second position (unlocked, first state of the mechanism unit MU). FIGS. 5 and 6 show the case where the arm 50 is in the first position (locked, second state of the mechanism unit MU).

It is indicated in FIGS. 3 to 6 that the number sleeve 26 comprises several recesses 54 or grooves 54 which correspond to the amount, set position and pitch of the possible dose units which can be set with the mechanism unit MU (e.g. 24 units). The arm 50 comprises a radially inwardly directed protrusion 53. The protrusion 53 is configured to engage into the recesses 54 in order to block a helical movement between the number sleeve 26 and the arm 50. As the arm 50 is rotationally and axially fixed to the inner body 10, this engagement will prevent a helical movement of the number sleeve 26 relative to the inner body 10.

As explained with respect to FIG. 2, setting and dispensing a drug dose is associated with a helical movement of the number sleeve 26. Therefore, with the arm 50 in the first position (see FIGS. 5 and 6), the block interface between the arm 50 and the number sleeve 26 prevents setting and dispensing a drug dose. The operational state of the mechanism unit MU is a locked state. When the arm 50 is in the second position (FIGS. 3 and 4), the block interface is released, setting and dispensing a drug dose is enabled and the operational state of the mechanism unit MU is an unlocked state.

FIGS. 3 and 5 further illustrate how the actuator 5 can be operated. The conductor path 44 is guided from the control unit 43A to the electromagnet 52. By sending an electric current through the conductor path 44 or by changing an electric current in the conductor path 44, the magnetization of the electromagnet 52 may be changed from repelling the magnet 51 to attracting the magnet 51 or vice versa. Controlling an electric current in the conductor path 44 may be done by the control unit 43A.

For example, the control unit 43A is configured to only enable operation or to only operate the actuator 5 by changing the current in the conductor path 44 and to thereby change the operation state of the mechanism unit MU (from the locked state to the unlocked state or vice versa), when an output signal from an electronic device 200 is received via the communication component 101 and forwarded to the control unit 43A. If no output signal is received, operation of the actuator 5 is prevented.

As an example, in FIGS. 5 and 6, the electromagnet 52 is not magnetized so that the electromagnet 52 and the magnet 51 do not magnetically interact. The flexible arm 50 is in the first position which may be its relaxed state. When operating the actuator 5, the electromagnet 52 is supplied with current and is then attracted by the magnet 51. The flexible arm 50 moves in radial outward direction into the second position (FIGS. 3 and 4). In this second position, the flexible arm 50 is pre-biased towards its first position. If the operation of the actuator 5 is interrupted by removing the current for the electromagnet 52, the flexible arm automatically returns into its first position.

FIGS. 7 to 12 show a third exemplary embodiment of the drug delivery device 100. The functionalities, especially concerning the setting and dispense mechanism and the communication component 101, may be essentially the same as for the previous exemplary embodiments. The actuator 5 for blocking and releasing dose setting is, however, different.

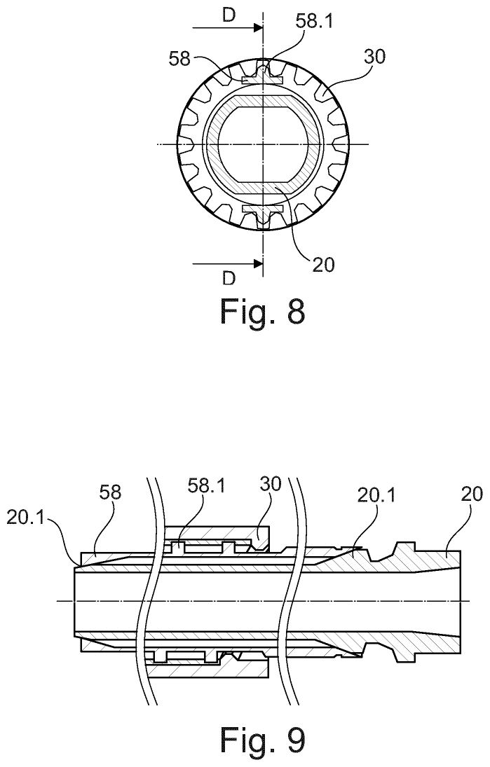

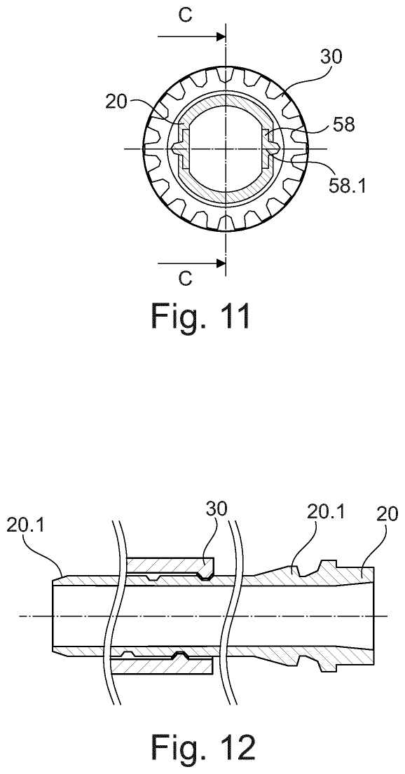

The mechanism unit MU comprises an intermediate element 58 in form of a blocking sleeve 58 which partially surrounds the distal drive sleeve 20. The blocking sleeve 58 comprises two elongated arms each with a wedge 58.1 protruding in radial outward direction (see FIGS. 8 and 11). The distal drive sleeve 20 comprises ramps 20.1. The actuator 5 comprises actuator elements 50 in form of actuator arms. The actuator arms 50 can be moved with help of an electric motor of the actuator 5. The actuator 5 is coupled to the drive sleeve coupler 22 and the actuator arms 50 are engaged with the blocking sleeve 58. A blocking sleeve spring 59 biases the blocking sleeve 58 in distal direction D.

FIG. 8 shows a view on the cross-sectional plane AA of FIG. 7. The last dose nut 30 comprises, on its inner surface, a number of recesses. For example, the number of recesses is equal to or a whole fraction of the number of dose steps in one dose setting revolution. FIG. 9 shows the view on the cross-sectional plane DD of FIG. 8.

FIGS. 7 to 9 show the drug delivery device 100 with the actuator arms 50 in a first position. The actuator arms 50 may remain in this first position after a short energization of the actuator 5. The actuator arms 50 in the first position have pulled and/or hold the blocking sleeve 58 in a lock position in which the blocking sleeve 58 is pulled over the ramps 20.1 of the distal drive sleeve 20. Thereby, the arms of the blocking sleeve 58 have been forced by the ramps 20.1 to move radially outwardly so that the wedges 58.1 engage into the recesses of the last dose nut 30. Thereby, a block interface is established which blocks relative rotation between the last dose nut 30 and the blocking sleeve 58. The blocking sleeve 58 is rotationally locked to the distal drive sleeve 20. Since the last dose nut 30 cannot rotate relative to the inner body 10, rotation of the distal drive sleeve 20 is prevented with the actuator arms 50 in the first position and the blocking sleeve 58 in the lock position. Thus, setting of a drug dose is prevented. As can be further seen in FIG. 7, with the blocking sleeve 58 in the lock position, the blocking sleeve spring 59 is compressed.

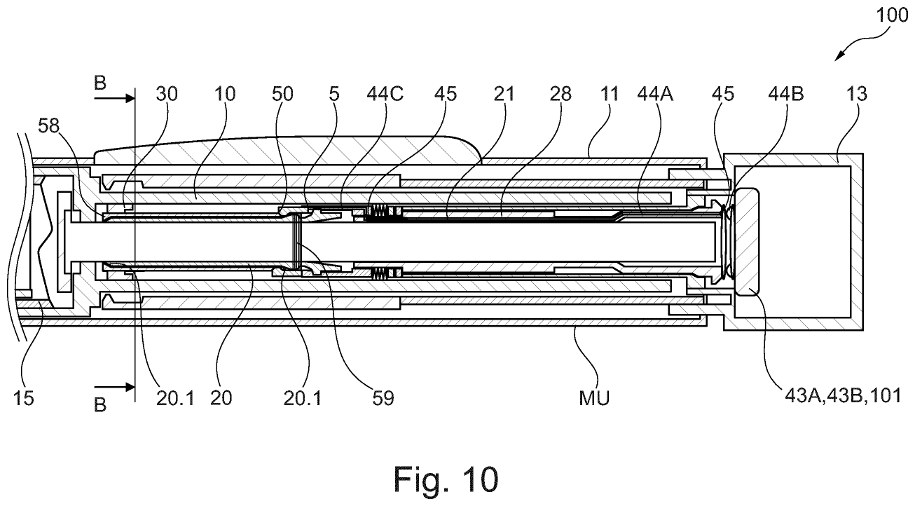

FIGS. 10 and 12 show the drug delivery device 100 with the actuator arms 50 in a second position in which they do no longer hold the blocking sleeve 58 in its lock position. FIG. 11 is the view on the cross-sectional plane BB of FIG. 20. FIG. 12 is the view on the cross-sectional plane CC of FIG. 21a.

The blocking sleeve spring 59 has pushed the blocking sleeve 58 in distal direction D so that the arms of blocking sleeve 58 are no longer held over the ramps 20.1 and can relax in radial inward direction into a release position. In the release position of the arms of the blocking sleeve 58, the wedges 58.1 of the blocking sleeve 58 do no longer engage into the recesses of the last dose nut 30 so that the block interface is released and rotation of the drive sleeve 20 relative to the last dose nut 30 is allowed. In this way, dose setting is enabled.

FIGS. 7 and 10 also show the electrical connection between the actuator 5 and the control unit 43A or the battery 43B, respectively. The conductor path 44 connecting the actuator 5 with the control unit 43A and/or the battery 43B comprises three sections 44A, 44B, 44C which are assigned to different elements of the drug delivery device 100. The control unit 43A, the battery 43B and the section 44B is fixed to the knob 13. The section 44A is fixed to the proximal drive sleeve 21. The section 44C is fixed to the drive sleeve coupler 22. An electrical connection between the sections 44A and 44B and 44A and 44C is maintained during dose dialing and/dose dispensing by contacts 45.

The operation of the actuator 5 may again be controlled by the control unit 43A. This may again be done dependent on whether an output signal of an electronic device 200 has been received via the communication component 101.

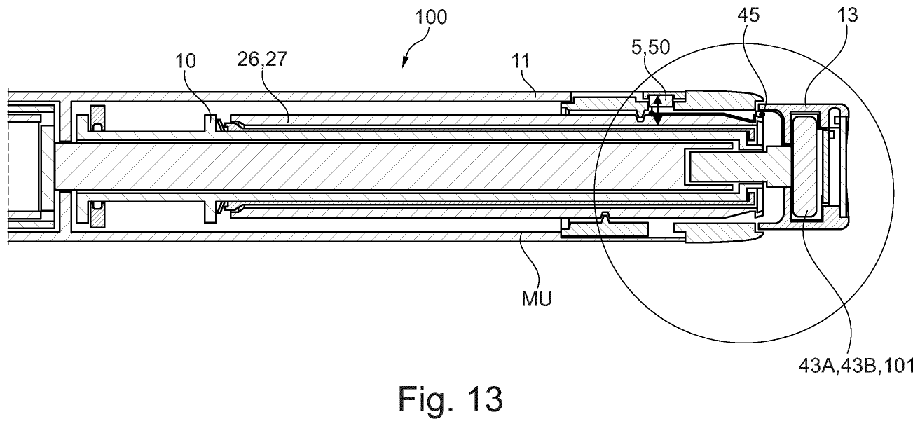

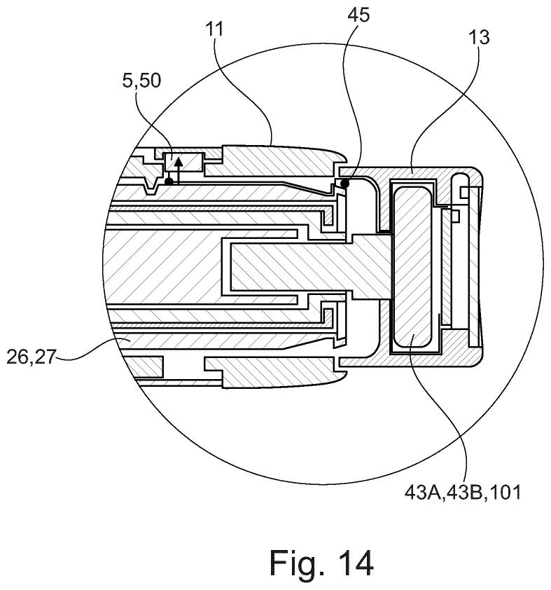

FIGS. 13 and 14 show a fourth exemplary embodiment of the drug delivery device 100. Again, this exemplary embodiment may have essentially the same functionalities, especially concerning the setting and dispense mechanism and the communication component 101, as the previous exemplary embodiments but deviates from the previous exemplary embodiments in the design of the actuator 5.

In the fourth exemplary embodiment, the control unit 43A, the battery 43B and the communication component 101 are coupled to the knob 13 so that they move together with the knob 13. The actuator 5 is coupled to the dial sleeve 27. The actuator element 50 of the actuator 5 is, e.g., a pin which can be moved in radial direction by the actuator 5.

In FIGS. 13 and 14 (FIG. 14 shows the circled region of FIG. 13) the actuator element 50 is in a first position in which it is engaged with the outer body 11. Due to this engagement, relative axial and rotational movement between the dial sleeve 27 and the outer body 11 is prevented. Consequently, dose setting and dose dispensing is prevented. When the actuator 5 is operated by the control unit 43A, the actuator element 50 may be moved in a second position in which it no longer engages with the outer body 11 so that dose setting and dose dispensing is enabled.

Also here, the operation of the actuator 5 may be controlled by the control unit 43A dependent on whether an output signal of an electronic device 200 has been received via the communication component 101.

Some or all of the actuators 5 described in connection with the previously described exemplary embodiment may also be combined.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N—(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof. An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The disclosure described herein is not limited by the description in conjunction with the exemplary embodiments. Rather, the disclosure comprises any new feature as well as any combination of features, particularly including any combination of features in the patent claims, even if said feature or said combination per se is not explicitly stated in the patent claims or exemplary embodiments.

### REFERENCE NUMERALS

5 actuator
10 inner body
11 outer body
12 window
13 knob
14 cap
15 cartridge holder
16 cartridge container
17 stopper
20 distal drive sleeve
20.1 ramp
21 proximal drive sleeve
22 drive sleeve coupler
23 distal clicker
24 proximal clicker
25 clutch spring
26 number sleeve
27 dial sleeve
28 clutch
29 plunger rod
30 last dose nut
43A control unit
43B battery
43C capacitor
44A first section of conductor path 44
44B second section of conductor path 44
44C third section of conductor path 44
45 sliding contact
50 actuator element
51 magnet
52 magnet
53 protrusion
54 recess
58 blocking sleeve
58.1 wedge
59 blocking sleeve spring
100 drug delivery device
101 communication component
200 electronic device
201 processor
202 communication component
203 communication component
204 charging component
300 external device
400 cloud service
MU mechanism unit
RU drug reservoir unit
D distal direction
P proximal direction
L longitudinal axis
R radial direction
C azimuthal direction/rotational direction/angular direction

1.-17. (canceled)

18. A method performable with an electronic device in order to communicate with a drug delivery device, the method comprising:
determining whether a user of the electronic device is authorized to operate the drug delivery device based on first information and second information, wherein:
the first information is indicative of a prescription of a drug to a patient, and
the second information is indicative of the drug the drug delivery device is foreseen to dispense, and
generating an output signal to be communicated to the drug delivery device in order to enable changing an operational state of the drug delivery device if the user is determined to be authorized to use the drug delivery device.

19. The method according to claim 18, wherein:
the first information is extracted from a first signal transmitted from an external device to the electronic device, and
the second information is extracted from a second signal transmitted from the drug delivery device to the electronic device.

20. The method according to claim 19, wherein:
at least one of the first signal or the second signal is authenticated before generating the output signal, and/or
at least one of the first signal or the second signal is cryptographically secured,
wherein extracting the first information and/or second information comprises decrypting at least one of the first signal or the second signal.

21. The method according to claim 19, wherein at least one of the first signal or the second signal is a wirelessly transmitted signal.

22. The method according to claim 21, wherein the second signal is a Near Field Communication signal.

23. The method according to claim 21, wherein the first signal is a Long Range Communication signal and/or is transmitted via a cloud service.

24. The method according to claim 18, further comprising:
generating a power signal in order transfer energy to the drug delivery device via induction.

25. The method according to claim 18, wherein:
the change of the operational state is associated with a mechanical change in a mechanism unit of the drug delivery device, and
the change of the operational state is a change between a state where setting a drug dose and/or dispensing a drug dose is prevented and a state where setting a drug dose and/or dispensing a drug dose is enabled.

26. The method according to claim 18, wherein the drug delivery device is an injection device.

27. An electronic device comprising one or more processors and one or more non-transitory computer readable media comprising instructions that, when executed by the one or more processors, cause the one or more processors to perform operations comprising:
determining whether a user of the electronic device is authorized to operate a drug delivery device based on first information and second information, wherein:
the first information is indicative for a prescription of a drug to a patient, and
the second information is indicative for the drug the drug delivery device is foreseen to dispense, and
generating an output signal to be communicated to the drug delivery device in order to enable changing an operational state of the drug delivery device if the user is determined to be authorized to use the drug delivery device.

28. The electronic device according to claim 27, further comprising:
one or more communication components configured to receive wireless signals and to send a wireless output signal.

29. The electronic device according to claim 27, further comprising:
at least one Near Field Communication component,
at least one Long Range Communication component, and
an inductive charging component.

30. One or more non-transitory computer readable media storing instructions that, when executed by an electronic device, cause the electronic device to perform the method according to claim 18.

31. The one or more non-transitory computer readable media of claim 30, wherein the drug delivery device is configured to retain a drug container with a drug or comprising a drug container with a drug.

32. A drug delivery device comprising:
a mechanism unit configured to be operatively coupled with a drug reservoir unit, wherein:
the mechanism unit is configured to enable a dispense process for dispensing a drug,
the mechanism unit comprises an arrangement for changing an operational state of the mechanism unit,
the mechanism unit comprises a communication component for receiving an output signal transmitted from an electronic device to the drug delivery device, and
the mechanism unit is configured such that operation of the arrangement in order to change the operational state of the mechanism unit is prevented unless an output signal from the electronic device is received via the communication component.

33. The drug delivery device according to claim 32, wherein
the drug delivery device is a device for self-administration,
the arrangement comprises an electromechanical actuator, and
operation of the arrangement for changing the operational state comprises operation of the electromechanical actuator.

34. The drug delivery device according to claim 32, wherein the electronic device is configured to perform operations comprising:
determining whether a user of the electronic device is authorized to operate the drug delivery device based on first information and second information, wherein:
the first information is indicative for a prescription of a drug to a patient, and
the second information is indicative for the drug the drug delivery device is foreseen to dispense, and
generating an output signal to be communicated to the drug delivery device in order to enable changing an operational state of the drug delivery device if the user is authorized to use the drug delivery device.

35. The drug delivery device according to claim 32, wherein the drug delivery device is configured to retain a drug container with a drug or comprising a drug container with a drug.

36. The drug delivery device according to claim 32, wherein the mechanism unit comprises a dispense mechanism for dispensing a drug dose, wherein the dispense mechanism comprises an energy member, wherein the energy member is configured to provide energy for moving a plunger rod in distal direction.
